(19) United States
(12) Patent Application Publication
(10) Pub. No.: US 2022/0214451 A1
(43) Pub. Date: Jul. 7, 2022

(54) SENSOR FUSION

(71) Applicant: Elliptic Laboratories AS, Oslo (NO)

(72) Inventor: Espen Klovning, Strømmen (NO)

(73) Assignee: Elliptic Laboratories AS, Oslo (NO)

(21) Appl. No.: 17/611,488

(22) PCT Filed: Jun. 8, 2020
(86) PCT No.: PCT/NO2020/050145

§ 371 Date: Nov. 15, 2021

(30) Foreign Application Priority Data
Jun. 7, 2019 (NO) 20190708

(51) Int. Cl. G01S 15/42; G01S 15/04; G01S 7/539; G06N 20/00

(52) U.S. Cl. CPC G01S 15/42; G06N 20/00; G01S 7/539; G01S 15/04

(57) ABSTRACT

The present teachings relate to a proximity detection system for an electronic device comprising a transmitter and a receiver, the transmitter being arranged to transmit a signal, at least some portion of which is directed towards an object, and the receiver being arranged to receive a reflected signal, the reflected signal being a portion of the signal reflected from the object, wherein the system comprises a first processing unit configured to, load and execute an engine for controlling the transmission of the signal, and extracting one or more parameters related to the object from the reflected signal; wherein the system further comprises a second processing unit configured to receive sensor data from other sensors in the electronic device; and transmit the sensor data to the engine, wherein the engine is configured to generate a proximity event by analyzing at least one of the one or more parameters, and at least some of the sensor data. The present teachings also relate a proximity detection system comprising a third processing unit, an electronic device comprising the proximity detection system, a method for generating a proximity event on an electronic device, and computer software product for implementing any method steps disclosed herein.

### TECHNICAL FIELD

The present teachings relate generally to sensor fusion, particularly to sensor fusion provided in portable electronic devices.

### BACKGROUND ART

Portable electronic devices such as smartphones are typically equipped with a proximity detection system. The proximity detection system is commonly infrared (“IR”) sensor based, but it can even be an acoustic sensor based system. Acoustic sensor based proximity detection systems commonly operate in the ultrasonic range of frequencies.

A main function of such a proximity sensor is to detect a condition when a user has positioned the electronic device close to their ear during an ongoing phone call, in which case the touchscreen of the device is disabled or switched off to prevent false touch events due to contact of the ear or other body part of the user with the screen of the mobile device. Since the touch screen is not normally used while the user is in call and has placed the device close to their head or next to their ear, the touch screen controller can either be switched off or may enter a low-power mode to save power. Additionally, the screen lighting of the device is also normally switched off to save power. The proximity detection system normally works by detecting an object within a field of view (“FoV”) of the proximity sensor. The FoV of a proximity sensor is a three-dimensional envelope or space around the proximity sensor within which the sensor can reliably detect a proximity event. In some applications, the proximity detection system can be used to recognize touchless gestures made by the object, i.e., gestures made in the air without coming in physical contact with the electronic device. Accordingly, the proximity detection system may compute one or more parameters related to the object. The object parameters may include position, distance, speed, estimated trajectory, and/or projected trajectory of the object.

In certain cases, a detection of a proximity event by the proximity detection system may trigger an execution of a certain undesired proximity response on the electronic device. An example of such an undesired proximity response is switching-off of the screen of the electronic device in response to a detection of a proximity event even though given the use case, the screen should not have been switched off. Such a situation may arise, for example, when the electronic device is in an in-call condition, but the device is not being held against an ear of the user. In such a condition, a detection of a proximity event, for example, due to the user's finger may trigger an undesired switching-off of the screen.

### SUMMARY

At least some problems inherent to the prior-art will be shown solved by the features of the accompanying independent claims.

The applicant has realized that conventional proximity detection systems may lack awareness of context or use case of the electronic device, which may lead to generation of undesired proximity response in the electronic device.

According to one aspect the context or use case of the electronic device may be estimated from one or more object parameters extracted by the proximity detection system.

According to another aspect, the one or more object parameters extracted by the proximity detection system may be processed in combination with sensor data from the other sensors in the electronic device for further improve the estimation of the context or use case of the electronic device.

The teachings may be applied in principle to most of the proximity detection systems, especially those based on transmission of a signal and reception of a return signal. Systems those are based on signals such as acoustic, electromagnetic radiation such as infrared (“IR”), light, magnetic field, and their likes fall within the ambit of the present teachings.

Viewed from a first perspective, there can be provided a proximity detection system for an electronic device, the proximity detection system comprising:
a transmitter; and
a receiver,
   the transmitter being arranged to transmit a signal, at least some portion of which signal is directed towards an object, and the receiver being arranged to receive a reflected signal, the reflected signal being a portion of the signal reflected from the object, wherein the system comprises a first processing unit configured to:
load and execute an engine for controlling the transmission of the signal; and
extract one or more parameters related to the object by analyzing the reflected signal, wherein
   the system further comprises a second processing unit configured to:
receive sensor data from other sensors in the electronic device; and transmit the sensor data to the engine, wherein
the engine is configured to generate a proximity event by analyzing:
at least one of said one or more parameters; and
at least some of the sensor data.

According to another aspect, the second processing unit is further configured to implement a virtual proximity sensor for interfacing the proximity event to an application programming interface (“API”). The API may be run on the electronic device or on another device.

It will be understood that the engine is software engine, or a computer software code that is used for controlling the signal, and extracting one or more parameters related to the object from the reflected signal is received by the receiver. Accordingly, the engine is configured to generate a proximity event by analyzing at least at least one parameter from the sensor data

According to another aspect, the first processing unit is configured to load and execute a first part of the engine, and the second processing unit is configured to load and execute a second part of the engine, and the first part of the engine is configured to extract one or more machine learning features from the reflected signal, the machine learning features being transmitted to the second processing unit, and the second part of the engine is configured to receive the sensor data and to generate a proximity event by analyzing at least one of the one or more machine learning features and at least some of the sensor data.

By doing this, a smaller amount of data needs to be exchanged between the first processing unit and the second processing unit, for example, in the form of packets on a shared bus, or packets on a serial interface, or even data placed in an area of memory that can be accessed by both processing units. According to an aspect, the processing can be performed more efficiently when the first processing unit is adapted to process acoustic data, while the second processing unit is adapted to process sensor data.

The signal, according to an aspect, is an acoustic signal. The signal is, according to another aspect, an ultrasonic signal.

More specifically, from a second perspective, there can also be provided a proximity detection system for an electronic device, the proximity detection system comprising:
a transmitter; and
a receiver,
   the transmitter being arranged to transmit a signal, at least some portion of which signal is directed towards an object, and the receiver being arranged to receive a reflected signal, the reflected signal being a portion of the signal reflected from the object, wherein the system comprises a first processing unit configured to:
load and execute a first part of an engine for controlling the transmission of the signal;
extract one or more parameters related to the object by analyzing the reflected signal; and
generate one or more machine learning features from at least one of said one or more parameters related to the object,
   wherein the system further comprises a second processing unit configured to:
receive sensor data from other sensors in the electronic device; and
receive the one or more machine learning features, wherein
   the second part of the engine is configured to generate a proximity event by analyzing:
at least one of the one or more machine learning features; and
at least some of the sensor data.

Viewed from a third perspective, there can also be provided a proximity detection system for an electronic device, the proximity detection system comprising:
a transmitter; and
a receiver,
   the transmitter being arranged to transmit a signal, at least some portion of which signal is directed towards an object, and the receiver being arranged to receive a reflected signal, the reflected signal being a portion of the signal reflected from the object, wherein the system comprises a first processing unit configured to:
load and execute a first part of an engine for controlling the transmission of the signal;
extract one or more parameters related to the object by analyzing the reflected signal; and
generate one or more machine learning features from at least one of said one or more parameters related to the object,
   wherein the system further comprises a second processing unit configured to:
receive sensor data from other sensors in the electronic device; and
transmit the sensor data to a third processing unit,
   wherein the third processing unit is configured to receive the one or more machine learning features; and
   the third part of the engine is configured to generate a proximity event by analyzing:
at least one of the one or more machine learning features; and
at least some of the sensor data, and wherein
   the third part of the engine is further configured to transmit the proximity event to the second processing unit.

By doing this, for example, when the signal is an ultrasound signal, the ultrasound features of the reflected signal are separated from the acoustic signal in the first processing unit, limiting the amount of data that is communicated with the third processing unit. As previously discussed, the ultrasound features may be communicated in the form of packets on a shared bus, or packets on a serial interface, or data placed in an area of memory that can be accessed by the first and third processing units.

According to another aspect viewed from any of the above perspectives, the second processing unit is also configured to implement a virtual proximity sensor for interfacing the proximity event to an application programming interface (“API”). The API may be run on the electronic device or on another device.

Viewed from a fourth perspective, there can also be provided a method for generating a proximity event on an electronic device comprising a transmitter, a receiver, a first processing unit, and a second processing unit, the method comprising:
Transmitting, via the transmitter, a signal towards an object; the transmission of the signal being controlled by an engine running on the first processing unit,
Receiving, at the receiver, a reflected signal, the reflected signal being a reflection of the signal reflected from the object
Analyzing, using the engine, the reflected signal;
Extracting at the engine, from the analysis of the reflected signal, one or more parameters related to the object;
Receiving, at the second processing unit, sensor data from other sensors in the electronic device
Transmitting the sensor data to the engine
Generating, via the engine, a proximity event by further analyzing the at least one of said one or more parameters in combination with at least some of the sensor data.

According to an aspect the signal is an acoustic signal. According to another aspect the signal is an ultrasonic signal.

Similarly, method for generating a proximity event on an electronic device comprising a transmitter, a receiver, a first processing unit, and a second processing unit according to other perspectives in this disclosure, e.g., using second and/or third perspectives can also be provided.

When viewed from yet another perspective, the present teachings can also provide a computer software product for implementing any of the method steps disclosed herein using a suitable processing means or processor. Accordingly, the present teachings also relate to a computer readable program code having specific capabilities for executing any method steps herein disclosed. In other words, the present teachings relate also to a non-transitory computer readable medium storing a program causing an electronic device to execute any method steps herein disclosed.

More specifically, for example, according to the first perspective, there can also be provided a computer software product which, when executed by a processor of an electronic device, causes the electronic device to:
execute an engine on a first processing unit;
transmit, via a transmitter, a signal towards an object, wherein the transmission of the signal is controlled by the engine;
receive, at a receiver, a reflected signal, the reflected signal being a reflection of the signal reflected from the object;
analyze the reflected signal;
extract from the analysis of the reflected signal, one or more parameters related to the object;
receive sensor data from other sensors in the electronic device
transmit the sensor data to the engine
generate a proximity event by further analyzing the at least one of said one or more parameters in combination with at least some of the sensor data.

The processor of the electronic device and the first processing unit may be the same device or they may be different devices.

As discussed previously the signal according to an aspect is an acoustic signal. The signal is according to another aspect an ultrasonic signal.

Similarly, a computer software product according to other perspectives in this disclosure, e.g., using second and/or third perspectives can also be provided.

It will be appreciated there can also be provided an electronic device comprising the proximity detection system discussed in this disclosure. Similarly, there can also be provided an electronic device configured to execute the method steps disclosed herein, and also an electronic device configured to execute the software product disclosed herein.

It will be appreciated that depending upon the use case, the object may be the user. In certain use cases, a body part of the user, such as a hand may be considered an object. Alternatively, if a user is considered an object, the hand may be considered as a part of the object. In other cases, the hand and the rest of the user's body may be considered different objects, given the range and/or sensitivity of the field of view of the transmitter/receiver combination. In some cases, an inanimate object such as a stylus or a pen may be considered as the object. The range and/or sensitivity may either be limited according to component specifications, or it may be statically or dynamically set to a certain value according to processing requirements.

It will be understood that at least some of the parameters related to the object may be extracted from the reflected signal relative to one or more characteristics of the signal. For example, for time of flight (“ToF”) measurements, a time-period between the transmitting of the signal and the reception of the reflected signal is measured. Accordingly, at least some processing done by the first processing unit for extracting one or more parameters related to the object from the reflected signal may be done relative to the signal transmitted by the transmitter.

The sensor data may comprise one or more of output data from sensors such as, accelerometer, gyro, inertial sensor, light sensor, camera, and microphone.

The signal may be a continuous signal, or it may be an intermittent signal. The signal may comprise either a single frequency or a plurality of frequencies. The signal may even comprise a single time limited transmission, or a series of time shifted transmissions of with equal or unequal frequencies and/or amplitudes. Time-period between the time shifted transmissions may be equal or unequal.

The proximity event may be either one or more of, a binary signal confirming presence of an object within the field of view of the proximity detection system, distance of the object from a given location on the electronic device, relative speed of the object with respect to the electronic device, trajectory of movement of the object, a projected or extrapolated trajectory of the object.

The signal is preferably an acoustic signal, more preferably an ultrasound signal. Accordingly, the transmitter is an ultrasound transmitter and the receiver an ultrasound receiver. According to yet another aspect, a plurality of different transmitters and/or receivers may be provided of the same type or different types, for example, a set of ultrasound transmitter and receivers, and a set of infrared (“IR”) transmitters and/or receivers such that the engine is configured to analyze signals received from a plurality of receivers.

Alternatively, or in addition, the teachings can also apply to other kinds of proximity detection systems such as those based on electric field, light, magnetic field that allow distance measurement.

As will be appreciated, the transmitter and receiver may either be different components or alternatively they can be the same transducer that is used in a transmit mode for transmitting the ultrasound signal and then in a receive mode for receiving the reflected ultrasound signal. If the transmitter and receiver are different components, they may be placed in the same location, or they may be installed at different locations on the electronic device. Furthermore, the electronic device may comprise a plurality of transmitters and/or a plurality of receivers. Multiple transmitter-receiver combinations may be used to extract spatial information related to the object and/or surroundings.

The teachings may involve computing a distance value by the processing, by the engine, of the reflected signal. Said distance value can be relative to the distance between the object and the electronic device.

The processing of the signal and the reflected signal is done by a processing unit such as a computer processor. The processing unit may either be the same processor that is used for processing signals received by a touchscreen of the electronic device, or it may be a separate processor. A usage of the term processing unit in this disclosure thus includes both alternatives, i.e., separate processors and same processor. The processing unit can be any type of computer processor, such as a DSP, an FPGA, or an ASIC. The processing unit may further comprise a memory and/or it may be operatively connected to a memory.

The range and/or sensitivity of the proximity detection system may either be limited according to component specifications, or it may be statically or dynamically adapted by the processing unit to a certain value according to processing requirements and/or use case of the electronic device.

The teachings also involve transmitting the proximity event to another electronic module of the electronic device. The proximity event may include one or more of: position, distance, speed, estimated trajectory, and projected trajectory. Another electronic module may be a hardware or software module, and may include any one or more of, application programming interface (“API”), and sensor fusion module.

In case of ultrasound signals, processing of the reflected signal or echo signal may be based on time of flight (“TOF”) measurements between the transmitted ultrasound signal and the corresponding received reflected signal. The processing of the echo signals may also be based on the amplitude of the measured signal, or phase difference between the signal and the reflected signal, or the frequency difference between the signal and the reflected signal, or a combination thereof. The signal may comprise either a single frequency or a plurality of frequencies. In another embodiment, the signal may comprise chirps.

The method steps are preferably implemented using a computing unit such as a computer or a data processor.

Viewed from yet another perspective, it can also be provided a computer software product for implementing any method steps disclosed herein. Accordingly, the present teachings also relate to a computer readable program code having specific capabilities for executing any method steps herein disclosed.

The term electronic device includes any device, mobile or stationary. Accordingly, devices such as mobile phones, smartwatches, tablets, notebook computers, desktop computers, and similar devices fall within the ambit of the term electronic device in this disclosure. Preferably, the electronic device is a smart speaker capable of providing a voice assistant service. The electronic device can be executing any of the method steps disclosed herein. Accordingly, any aspects discussed in context of the method or process also apply to the product aspects in the present teachings.

To summarize, the present teachings relate to a proximity detection system for an electronic device comprising a transmitter and a receiver, the transmitter being arranged to transmit a signal, at least some portion of which is directed towards an object, and the receiver being arranged to receive a reflected signal, the reflected signal being a portion of the signal reflected from the object, wherein the system comprises a first processing unit configured to, load and execute an engine for controlling the transmission of the signal, and extracting one or more parameters related to the object from the reflected signal; wherein the system further comprises a second processing unit configured to receive sensor data from other sensors in the electronic device; and transmit the sensor data to the engine, wherein the engine is configured to generate a proximity event by analyzing at least one of the one or more parameters, and at least some of the sensor data.

The present teachings also relate a proximity detection system comprising a third processing unit, an electronic device comprising the proximity detection system, a method for generating a proximity event on an electronic device, and computer software product for implementing any method steps disclosed herein.

Example embodiments are described hereinafter with reference to the accompanying drawings.

### BRIEF DESCRIPTION OF DRAWINGS

FIG. 1 shows a block diagram of an acoustic proximity detection system

FIG. 2 shows a block diagram of an acoustic proximity detection system comprising a second processing unit

FIG. 3 shows another block diagram of an acoustic proximity detection system comprising a second processing unit

FIG. 4 shows a block diagram of an acoustic proximity detection system comprising a third processing unit

### DETAILED DESCRIPTION

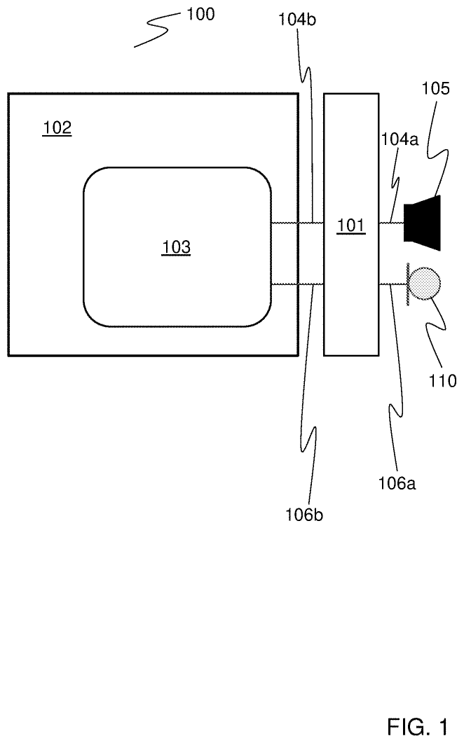

FIG. 1 illustrates a block diagram of a proximity detection system 100. The proximity detection system 100 is of acoustic type. The system 100 comprises a transmitting means such as a speaker 105 and a receiving means 110 such as microphone. The transmitting means 105 is used for transmitting an acoustic signal, whereas the receiving means 110 is used for receiving a reflection of the acoustic signal transmitted by the transmitting means 105. The acoustic signal is preferably in the ultrasonic range or it is an ultrasound signal. The transmitting and receiving means can be separate components or they may be the same transducer operated as a transmitter and then as a receiver. In some cases, the transmitting means 105 may be the same speaker of the electronic device that is used for playback of audio signals, such as music. In some cases, the receiving means 110 may be the same microphone of the electronic device that is used for receiving of audio signals, such as voice of the user. Alternatively, the transmitting means 105 and/or the receiving means 110 may be dedicated components used only for transmitting and receiving ultrasound signals. In some cases, the transmitting means 105 may comprise a plurality of transmitters of the same or different types. In some cases, the receiving means 110 may comprise a plurality of receivers of the same or different types.

The acoustic signal may be a plurality of signals. The signal may be continuous or intermittent.

The transmitter 105 and receiver 110 are shown connected, through signal paths 104a and 106a respectively, to an audio codec 101 which may, for example, be a WCD codec specified by Qualcomm®. The audio codec 101 is connected, through signal paths 104b and 106b, to a first processing unit 102, such as a digital signal processor (“DSP”) which may, for example, be a Hexagon™ DSP by Qualcomm®. The first processing unit 102 is configured to execute a code or engine 103 for processing the acoustic signals.

Each of the signal paths 104a, 104b, 106a, 106b may either be a single line or a bus, serial or parallel. Any of the paths may be direct signal paths, or they may be indirect, such as one or more shared memory locations accessible by the blocks within which the respective path 104a, 104b, 106a, 106b is shown.

For simplicity, the terms processing unit such as the first processing unit 102 and DSP are used interchangeably in this disclosure. It will be appreciated that the first processing unit 102 may also be realized using a microprocessor, a microcontroller or the like having at least one processing core. Any analogue signal processing blocks may either be located on the same chip with the at least one processing core, or the processing system may be realized as a System on Chip (“SoC”), a Multichip module (“MCM”), or even an Application Specific Integrated Circuit (“ASIC”). Furthermore, the codec 101 and the first processing unit 102 may be the same hardware component or different components.

In proximity detection mode of operation, the software code 103 running on the processing unit 102 sends instructions to the codec 101 for transmitting an ultrasound signal through the transmitter 105. The ultrasound signal generated by the code 103 can take many different forms, for example, frequency, signal envelope, amplitude, periodicity, etc. The form may be set by the user or preferably automatically by the use case or operation scenario of the electronic device.

In addition, the engine 103 may be controlled via an application programming interface (“API”) (not shown in the figure). The ultrasound control API may provide an interface to the engine 103 such that one or more of the parameters related to the proximity system may be set as desired. For example, in the simple case where the engine 103 is arranged to provide an ultrasound signal with a single, selectable frequency, the API provides a mechanism for the programmer to choose the frequency. Similarly, where the engine 103 is arranged to provide a multi-frequency ultrasound signal, the frequencies and/or the relative amplitudes of the various frequency components may be programmed by the using the API. However, there are typically many more parameters regarding the ultrasound signal that may be set by the API.

The receiver 110 is configured to receive a reflected acoustic signal. The reflected acoustic signal is the signal that has propagated towards the receiver 110 after being reflected by an object. The receiver 110 generates an electrical signal in response to the received acoustic signal. The electrical signal is then passed to the codec 101 and the processing unit 102. The code 103 extracts at least one parameter of interest from the electrical signal. The parameters of interest include, time-of-flight (“TOF”) of the acoustic signals, i.e., time difference between the transmitted acoustic signal and the received acoustic signals, doppler shift, phase shifts, amplitude variations, convolutions, additions, subtractions, etc.

Based on one or more of the extracted parameters of interest from one or more of the acoustic signals, the code 103 may determine if an object is present within a FoV of the system, e.g., within a given distance of the receiver 110. From the one or more of the electrical signals, the code 103 may further calculate information such as a distance, speed, acceleration and/or trajectory of the object.

The code 103 may further comprise a machine learning (“ML”) module that is used to improve the determination of the use case of the device. The ML module can help reduce undesired responses to an object being detected within the FoV of the system. An example of such an undesired response involving screen switching-off was provided earlier in this disclosure.

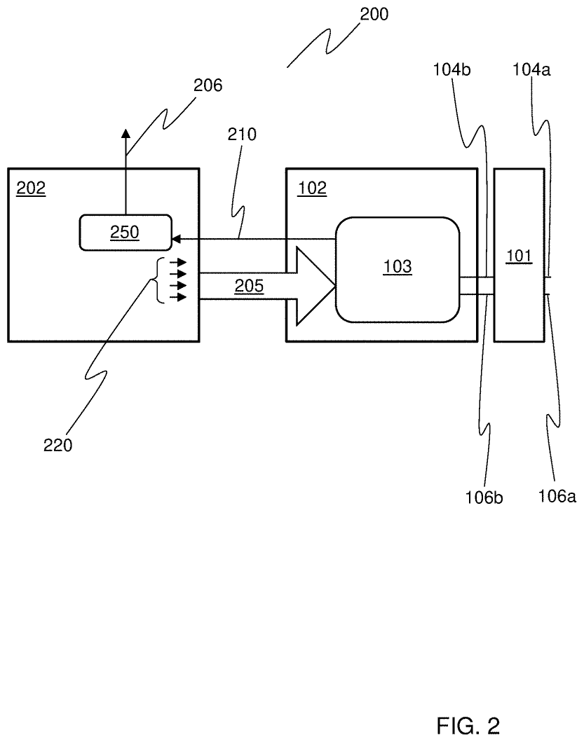

FIG. 2 shows a first variation 200 of the proximity detection system. The transmitting means 105 and receiving means 110 are not explicitly shown, but the signal paths 104a, 104b, 106a, 106b are visible. In this variation 200, the system further includes a second processing unit shown here as a sensor hub module 202. The sensor hub module 202 handles sensor data 220 from a plurality of sensors in the electronic device. The second processing unit 202 has access to sensor data 220 from one or more of sensors such as accelerometer, gyro, inclinometer, compass, light sensor, camera, hall sensor, microphone, etc. At least some of the sensor data 220 is transmitted as sensor output signals 205 to the ultrasound engine 103, for example, through a bus or other suitable transmitting means. Accordingly, second processing unit 202 is configured to receive sensor data 220 from other sensors in the electronic device, and at least some of the sensor data 220 is transmitted to the engine. In some cases, at least some of the sensor data 220 may be received by the ultrasound engine 103 from another software or hardware module, or directly from a sensor of the electronic device, instead of the at least some of the sensor data being received from the sensor hub module 202. For example, the touch controller may transmit self-capacitance data directly to the ultrasound engine 103.

Within the sensor hub module 202, a virtual proximity sensor 250 can be implemented. In this case, the virtual proximity sensor 250 receives ultrasound proximity data 210 from the ultrasound engine 103. Instead of communicating directly with the ultrasound engine 103, the API communicates with the virtual proximity sensor 250 from which the API receives proximity data 206. The proximity data 206 can either be a copy of the ultrasound proximity data 210, or it may be a processed version of the ultrasound proximity data 210. It will be understood that an occurring proximity event is communicated by the engine 103 via the ultrasound proximity data 210. Accordingly, the engine 103 is configured to generate a proximity event by analyzing at least one of the parameters related to the object extracted by analyzing the reflected signal received by the receiver 110, and at least some of the sensor data 220. If the virtual proximity sensor 250 is implemented, the proximity event can then be passed on directly, or after further processing, in the proximity data 206. As mentioned previously, the proximity data 206 can be a copy of the ultrasound proximity data 210, for example if the virtual proximity sensor 250 is not implemented.

In another case (not shown explicitly in FIG. 2), the virtual proximity sensor 250 is implemented in another module, such as a sensor hardware abstraction layer (“HAL”). In this case, the ultrasound proximity data 210 is sent by the ultrasound engine 103 directly or indirectly to the sensor HAL.

The sensor hub module 202 may be a separate processor or DSP, or it may be a part of the processing unit 102 in terms of hardware.

In most cases, at least some the sensor output signals 205 are transmitted at a rate of at least 10 Hz. According to another aspect, the transmission rate of the sensor data 205 is between 20 Hz to 50 Hz. According to another aspect, the transmission rate is 120 Hz. In some cases, at least some of the sensor output signals 205 are transmitted whenever their corresponding sensor data 220 changes more than a predetermined limit. Given the capability of the system, hardware or software, a higher transmission rate may be preferable to provide further resolution of events occurring in the sensor data. As it will be appreciated, power consumption requirement will be another aspect that may determine the transmission rate suitable for a given application.

The data rate of the sensor output signals 205 may not be the same as the data rate of the proximity data being handled in the engine 103, in which case the engine 103 is configured to handle the date rate difference, for example, by normalizing, upsampling, or downsampling the sensor data 205 relative to the proximity data being handled in the engine 103. In some cases, the engine 103 also comprises a machine learning module.

As it will be appreciated, the engine 103 as proposed in this case may not only detect if an object is present within the FoV of the system, but also can interpret the sensor data 205. The sensor data 205 may be raw data from the sensors, or it can be features or parameters from preprocessed data from sensors such as touch controller, accelerometer, and gyroscope. Accordingly, the engine 103 is able to further determine the context or use case of the electronic device. In some cases, the raw sensor data received by the engine 103 directly fed to the machine learning module for further processing.

Upon evaluating the context, the engine 103 sends ultrasound proximity data to 210 to the virtual proximity sensor 250. The ultrasound proximity data 210 comprises data related to the object evaluated by the engine 103 in context of the sensor data 205. Accordingly, the engine 103 may prevent false or undesirable proximity events. This may happen, for example, when the information from the audio codec 101 indicates that something is covering the speaker and microphone subsystems, but that since the device is placed on a table, the system's screen should not be switched off. From the sensor data 205 (which may contain accelerometer data, providing information about the orientation of the phone relative to the gravitational pull of the earth), the engine 103 can determine that the device is resting on a table, and that the screen should not be turned off as a result.

It will be appreciated that specific terms such as ultrasound signal, ultrasound features, ultrasound proximity data are used just as examples for the ease of discussion. If another kind of proximity system or principle is used, for example, IR, such terms may in such case correspond to the IR signal, IR proximity data and so forth.

As with rest of the features of FIG. 1, in the first variation shown in FIG. 2, the code 103 may further comprise a machine learning (“ML”) module, as outlined also previously, that is used to improve the determination of the use case of the device.

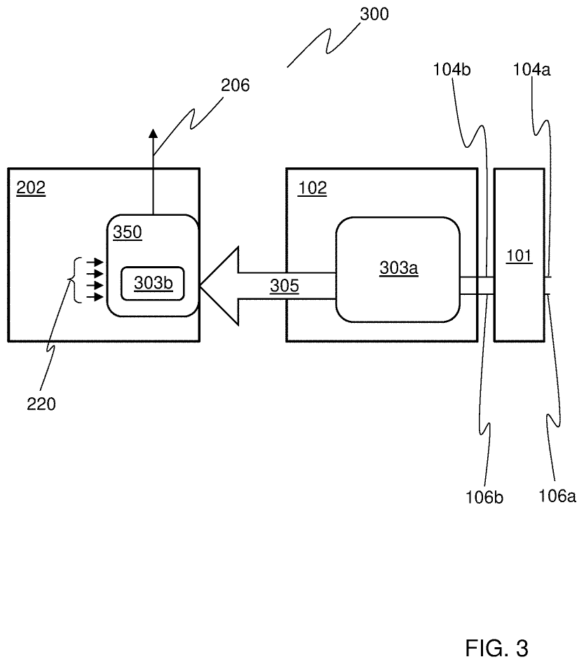

FIG. 3 shows a second variation 300 of the proximity detection system. The transmitting means 105 and receiving means 110 are not explicitly shown, but the signal paths 104a, 104b, 106a, 106b are still visible. In this variation 300, the engine is split in two parts, the first part 303a of the engine runs a frontend code on the first processing unit 102. As will be appreciated, the frontend 303a processes the signals related to the transmitting means 105 and receiving means 110. The frontend 303a transmits machine learning (“ML”) features 305, such as distance value, signal strength, etc., to a proximity fusion module 350 in the sensor hub 202. The machine learning features 305 are further processed in a second part 303b of the engine or the backend. The backend 303b may comprise a machine learning module. As discussed previously, the sensor hub module 202 or the second processing unit, has access to sensor data 220 from other sensors such as accelerometer, gyro, inclinometer, compass, light sensor, etc. The sensor data 220 is provided to the machine learning module 303b. The data rate from various sensors may not be the same as the data transmission rate of the ML features 305, in which case the backend 303b is configured to handle the data rate difference, for example, by normalizing, upsampling, and/or downsampling the appropriate ML features 305 data or the other sensor data 220.

In some cases, at least some of the sensor data 220 may be received by the second part 303b of the engine from another software or hardware module, or directly from a sensor of the electronic device, instead of the at least some of the sensor data being received from a module in the sensor hub module 202. For example, touch controller may transmit self-capacitance data directly to the second part 303b of the engine.

As will be appreciated, in comparison with FIG. 2, in the second variation 300, the sensor data 220 is not required to be transmitted to the first processing unit 102. Instead, the sensor data 220 can be handled within the sensor hub 202 itself. Further in contrast to the virtual proximity sensor 250, the proximity fusion module 350 can be an enhanced virtual sensor with processing capability. Since data is prevented from being transmitted back and forth between the sensor hub 202 and the first processing unit 102 in the second variation 300, the proximity detection system can be made faster. In addition, power saving can also be achieved. Furthermore, hardware requirements for the processing unit 102 can be relaxed, and more even distribution of processing load achieved.

The machine learning features 305 are transmitted at a rate of at least 10 Hz. According to another aspect, the transmission rate of the machine learning features 305 is between 20 Hz to 120 Hz. According to another aspect, the transmission rate of the machine learning features 305 is between 60 Hz to 120 Hz. According to another aspect, the transmission rate is between 20 Hz to 50 Hz. According to yet another aspect, the transmission rate is 120 Hz. Given the capability of the system, hardware or software, a higher transmission rate of the machine learning features 305 may be preferable to provide further resolution of events occurring in the proximity system. As it will be appreciated, power consumption requirement will be another aspect that will determine the transmission rate suitable for a given application.

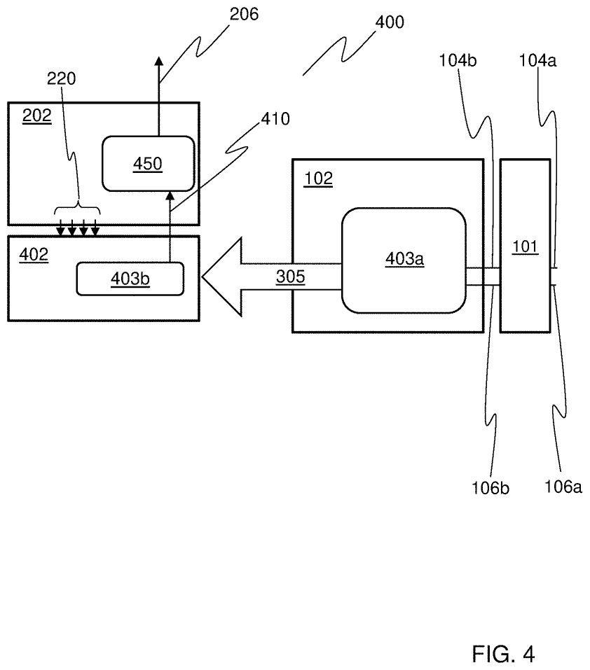

FIG. 4 shows a third variation 400 of the proximity detection system. The transmitting means 105 and receiving means 110 are not explicitly shown, but the signal paths 104a, 104b, 106a, 106b are visible. In this variation 400, the engine is split in two parts, the first part 403a of the engine runs a frontend code on the first processing unit 102. As will be appreciated, the frontend 403a processes the signals related to the transmitting means 105 and receiving means 110. The frontend 403a transmits machine learning features 305 to a third processing unit 402, which is shown as an artificial intelligence (“AI”) module 402. The machine learning features 305 are further processed in the second part 403b of the engine or the backend. The backend may also apply machine learning data processing on the machine learning features 305. The backend 403b may also comprise a machine learning module. In principle, the frontend 403a and backend 403b may be similar as those 303a and 303b in the second variation 300, or they may redistribute the signal processing differently, or perform additional functions.

In some cases, at least some of the sensor data 220 may be received by the second part 403b of the engine from another software or hardware module, or directly from a sensor of the electronic device, instead of the at least some of the sensor data being received from the sensor hub module 202. For example, touch controller may transmit self-capacitance data directly to the second part 303b of the second part 403b of the engine.

As discussed previously, the second processing unit 202 or the sensor hub module 202 has access to sensor data 220 from other sensors such as accelerometer, gyro, inclinometer, compass, light sensor, etc. The sensor data 220 is provided to the backend 403b through the Al module 402.

The AI module 402 may be a separate hardware, such as a dedicated chip or integrated circuit (“IC”), or it may be a part of the second processing unit or sensor hub 202. As may be appreciated, if AI module is a dedicated application specific integrated circuit (“ASIC”), it may be realized as a device optimized for performing processing on the ML features 305. The processing capacity of the AI module 402 may be adjusted according to the processing requirements on the ML features, for example, as communicated by the frontend 403a, and/or by the API.

The backend 403b is configured to send processed proximity data 410 to a virtual proximity sensor 450 implemented in the processing unit 202. The API communicates with the virtual proximity sensor 450 from which the API receives proximity data 206. The proximity data 206 can either be a copy of the processed proximity data 410, or it may be a further processed version of the processed proximity data 410.

As with common features of the presented variations, the machine learning features 305 are transmitted, here also, at a rate of at least 10 Hz. According to another aspect, the transmission rate of the machine learning features 305 is between 20 Hz to 120 Hz. According to another aspect, the transmission rate of the machine learning features 305 is between 60 Hz to 120 Hz. According to another aspect, the transmission rate is between 20 Hz to 50 Hz. According to yet another aspect, the transmission rate is 120 Hz. Given the capability of the system, hardware or software, a higher transmission rate of the machine learning features 305 may be preferable to provide further resolution of events occurring in the proximity system. As it will be appreciated, power consumption requirement will be another aspect that will determine the transmission rate suitable for a given application.

This architecture has an advantage of distributing the tasks according to specialized features of each processing unit so as to enhance performance. For example, the audio DSP processes data from the reflected signal, the sensor hub processes data from the sensors, and the Al modules processes the features provided by the audio DSP and the sensor hub. In addition, the transmission of features, instead of raw data, requires the transmission of smaller amounts of data between the processing units.

In addition, all the variations presented above, the backend may further be provided touchscreen controller data.

Various embodiments have been described above for a proximity detection system, an electronic device comprising any of the proximity detection systems, a method for proximity detection or a method for generating a proximity event, and a computer software product for at least partially implementing the method. Those skilled in the art will understand, however that changes and modifications may be made to those examples without departing from the spirit and scope of the following claims and their equivalents. It will further be appreciated that aspects and/or features from the method and product embodiments discussed herein may be freely combined.

Certain embodiments of the present teachings are summarized in the following clauses.

Clause 1.

A proximity detection system for an electronic device, the system comprising:
a transmitter, and
a receiver;
the transmitter being arranged to transmit a signal, at least some portion of which is directed towards an object, and the receiver being arranged to receive a reflected signal, the reflected signal being a portion of the signal reflected from the object, wherein the system comprises a first processing unit configured to:
   load and execute an engine for controlling the transmission of the signal, and
   extracting one or more parameters related to the object from the reflected signal; wherein
      the system further comprises a second processing unit configured to:
receive sensor data from other sensors in the electronic device; and
transmit the sensor data to the engine, wherein
   the engine is configured to generate a proximity event by analyzing at least one of the one or more parameters, and at least some of the sensor data.

Clause 2.

Proximity detection system according to clause 1, wherein the transmitter and the receiver are a common component, wherein the component is configured to:
transmit the signal when functioning as the transmitter; and
receive the reflected signal when functioning as the receiver.

Clause 3.

Proximity detection system according to any of the above clauses, wherein the signal is an ultrasound signal, and the transmitter and the receiver are an ultrasound transmitter and an ultrasound receiver respectively.

Clause 4.

Proximity detection system according to any of the above clauses, wherein the sensor data comprises one or more of outputs from sensors such as, accelerometer, gyro, inertial sensor, light sensor, camera, and microphone.

Clause 5.

Proximity detection system according to any of the above clauses, wherein the proximity event is either one or more of, a binary signal confirming presence of an object within the field of view of the proximity detection system, distance of the object from a given location on the electronic device, relative speed of the object with respect to the electronic device, trajectory of movement of the object, or a projected or extrapolated trajectory of the object.

Clause 6.

Proximity detection system according to any of the above clauses, wherein the second processing unit is also configured to implement a virtual proximity sensor for interfacing the proximity event to an application programming interface (“API”).

Clause 7.

Proximity detection system according to any of the above clauses, wherein
the first processing unit is configured to load and execute a first part of the engine, and the second processing unit is configured to load and execute a second part of the engine, and
the first part of the engine is configured to extract one or more machine learning features from the reflected signal, the machine learning features being transmitted to the second processing unit, and
the second part of the engine is configured receive the sensor data, and to generate a proximity event by analyzing at least one of the one or more machine learning features and at least some of the sensor data.

Clause 8.

A proximity detection system for an electronic device, the system comprising:
a transmitter, and
a receiver;
the transmitter being arranged to transmit a signal, at least some portion of which is directed towards an object, and the receiver being arranged to receive a reflected signal, the reflected signal being a portion of the signal reflected from the object, wherein the system comprises a first processing unit configured to:
   load and execute a first part of an engine for controlling the transmission of the signal;
   extract one or more parameters related to the object from the reflected signal; and
   generate one or more machine learning features from at least one of the one or more parameters related to the object; wherein
      the system further comprises a second processing unit configured to:
receive sensor data from other sensors in the electronic device, and
receive the one or more machine learning features; wherein
   the second part of the engine is configured to generate a proximity event by analyzing at least one of the one or more machine learning features, and at least some of the sensor data.

Clause 9.

A proximity detection system for an electronic device, the system comprising:
a transmitter, and
a receiver;
the transmitter being arranged to transmit a signal, at least some portion of which is directed towards an object, and the receiver being arranged to receive a reflected signal, the reflected signal being a portion of the signal reflected from the object, wherein the system comprises a first processing unit configured to:
load and execute a first part of an engine for controlling the transmission of the signal,
extract one or more parameters related to the object from the reflected signal, and
generate one or more machine learning features from at least one of the one or more parameters related to the object; wherein
   the system further comprises a second processing unit configured to:
receive sensor data from other sensors in the electronic device, and transmit the sensor data to a third processing unit; wherein
   the third processing unit further configured to receive the one or more machine learning features, and wherein
   the third part of the engine is configured to generate a proximity event by analyzing at least one of the one or more machine learning features and at least some of the sensor data, and to transmit the proximity event to the second processing unit.

Clause 10.

A method for generating a proximity event on an electronic device, the electronic device comprising a transmitter and a receiver, the method comprising:
Transmitting, via the transmitter, a signal towards an object; the transmission of the signal being controlled by an engine running on the first processing unit,
Receiving, at the receiver, a reflected signal, the reflected signal being a reflection of the signal reflected from the object
Analyzing, using the engine, the reflected signal;
Extracting at the engine, from the analysis of the reflected signal, one or more parameters related to the object;
Receiving, at the second processing unit, sensor data from other sensors in the electronic device
Transmitting the sensor data to the engine
Generating, via the engine, a proximity event by further analyzing the at least one of said one or more parameters in combination with at least some of the sensor data.

Clause 11.

A computer software product which, when executed by a processor of an electronic device, causes the electronic device to:
execute an engine on a first processing unit;
transmit, via a transmitter, a signal towards an object, wherein the transmission of the signal is controlled by the engine;
receive, at a receiver, a reflected signal, the reflected signal being a reflection of the signal reflected from the object;
analyze the reflected signal;
extract from the analysis of the reflected signal, one or more parameters related to the object;
receive sensor data from other sensors in the electronic device
transmit the sensor data to the engine
generate a proximity event by further analyzing the at least one of said one or more parameters in combination with at least some of the sensor data.

Clause 12.

An electronic device comprising the proximity detection system of any of the clauses 1-9.

1. A proximity detection system for an electronic device, the proximity detection system comprising:
a transmitter;
a receiver;
wherein the transmitter is arranged to transmit a signal, at least some portion of which is directed towards an object, and the receiver being arranged to receive a reflected signal, the reflected signal being a portion of the signal reflected from the object, wherein the system comprises a first processing unit configured to:
load and execute an engine for controlling the transmission of the signal; and
extracting extract one or more parameters related to the object from the reflected signal;
a second processing unit configured to:
receive sensor data from other sensors in the electronic device; and
transmit the sensor data to the engine; and
wherein the engine is configured to generate a proximity event by analyzing at least one of the one or more parameters, and at least some of the sensor data.

2. The proximity detection system according to claim 1, wherein:
the transmitter and the receiver are a common component; and
wherein the component is configured to:
transmit the signal when functioning as the transmitter; and
receive the reflected signal when functioning as the receiver.

3. The proximity detection system according to claim 1, wherein the signal is an ultrasound signal, and the transmitter and the receiver are an ultrasound transmitter and an ultrasound receiver respectively.

4. The proximity detection system according to claim 1, wherein the sensor data comprises one or more of outputs from sensors such as, accelerometer, gyro, inertial sensor, light sensor, camera, and microphone.

5. The proximity detection system according to claim 1, wherein the proximity event comprises at least one of, a binary signal confirming presence of an object within the field of view of the proximity detection system, distance of the object from a given location on the electronic device, relative speed of the object with respect to the electronic device, trajectory of movement of the object, and a projected or extrapolated trajectory of the object.

6. The proximity detection system according to claim 1, wherein the second processing unit is configured to implement a virtual proximity sensor for interfacing the proximity event to an application programming interface (“API”).

7. The proximity detection system according to claim 1, wherein:
the first processing unit is configured to load and execute a first part of the engine;
the second processing unit is configured to load and execute a second part of the engine;
the first part of the engine is configured to extract one or more machine learning features from the reflected signal, the machine learning features being transmitted to the second processing unit; and
the second part of the engine is configured receive the sensor data, and to generate a proximity event by analyzing at least one of the one or more machine learning features and at least some of the sensor data.

8. A proximity detection system for an electronic device, the proximity detection system comprising:
a transmitter;
a receiver;
wherein the transmitter is arranged to transmit a signal, at least some portion of which is directed towards an object, and the receiver being arranged to receive a reflected signal, the reflected signal being a portion of the signal reflected from the object;
a first processing unit configured to:
load and execute a first part of an engine for controlling the transmission of the signal;
extract one or more parameters related to the object from the reflected signal; and
generate one or more machine learning features from at least one of the one or more parameters related to the object; wherein
a second processing unit configured to:
receive sensor data from other sensors in the electronic device; and
receive the one or more machine learning features; and
wherein the second part of the engine is configured to generate a proximity event by analyzing at least one of the one or more machine learning features, and at least some of the sensor data.

9. A proximity detection system for an electronic device, the proximity detection system comprising:
a transmitter;
a receiver;
wherein the transmitter is arranged to transmit a signal, at least some portion of which is directed towards an object, and the receiver being arranged to receive a reflected signal, the reflected signal being a portion of the signal reflected from the object,
a first processing unit configured to:
load and execute a first part of an engine for controlling the transmission of the signal; and
extract one or more parameters related to the object from the reflected signal; and
generate one or more machine learning features from at least one of the one or more parameters related to the object;
wherein the system further comprises a second processing unit configured to:
receive sensor data from other sensors in the electronic device; and
transmit the sensor data to a third processing unit;
wherein the third processing unit is configured to receive the one or more machine learning features; and
wherein the third part of the engine is configured to generate a proximity event by analyzing at least one of the one or more machine learning features and at least some of the sensor data, and to transmit the proximity event to the second processing unit.

10. A method for generating a proximity event on an electronic device, the electronic device comprising a transmitter and a receiver, the method comprising:
transmitting, via the transmitter, a signal towards an object; the transmission of the signal being controlled by an engine running on the first processing unit;
receiving, at the receiver, a reflected signal, the reflected signal being a reflection of the signal reflected from the object;
analyzing, using the engine, the reflected signal;
extracting at the engine, from the analysis of the reflected signal, one or more parameters related to the object;
receiving, at the second processing unit, sensor data from other sensors in the electronic device;
transmitting the sensor data to the engine; and
generating, via the engine, a proximity event by further analyzing the at least one of said one or more parameters in combination with at least some of the sensor data.

11. A computer-program product comprising a non-transitory computer-usable medium having computer-readable program code embodied therein, the computer-readable program code adapted to be executed to implement a method comprising:
executing an engine on a first processing unit;
transmitting, via a transmitter, a signal towards an object, wherein the transmission of the signal is controlled by the engine;
receiving, at a receiver, a reflected signal, the reflected signal being a reflection of the signal reflected from the object;
analyzing the reflected signal;
extracting from the analysis of the reflected signal, one or more parameters related to the object;
receiving sensor data from other sensors in the electronic device;
transmitting the sensor data to the engine; and
a proximity event by further analyzing the at least one of said one or more parameters in combination with at least some of the sensor data.

12. An electronic device comprising the proximity detection system of claim 1.

13. The proximity detection system according to claim 4, wherein the sensors comprise accelerometer, gyro, inertial sensor, light sensor, camera, and microphone.
